(19) United States
(12) Patent Application Publication
(10) Pub. No.: US 2015/0342836 A1
(43) Pub. Date: Dec. 3, 2015

(54) Imaging Feeding Tube with Illumination Control

(71) Applicant: Covidien LP, Mansfield, MA (US)

(72) Inventor: Robert Allyn, Pacific, MO (US)

(21) Appl. No.: 14/721,190

(22) Filed: May 26, 2015

Related U.S. Application Data
(60) Provisional application No. 62/003,587, filed on May 28, 2014.

(51) Int. Cl. A61J 15/00; A61B 1/06; A61B 1/045; A61B 1/005; A61B 1/12; A61B 1/05; A61B 1/00

(52) U.S. Cl. CPC A61J 15/0088; A61B 1/05; A61B 1/0676; A61B 1/045; A61B 1/00055; A61B 1/00006; A61B 1/005; A61B 1/00119; A61B 1/00128; A61B 1/00062; A61B 1/128; A61J 15/0003

(57) ABSTRACT

A feeding tube assembly having a flexible feeding tube with an imaging assembly including an illumination source configured to illuminate an ambient environment of the imaging assembly and an imaging device configured to generate and transmit imaging signals indicative of images of an alimentary canal of a subject is disclosed. The feeding tube assembly has controller in communication with the illumination source wherein the controller is configured to energize the illumination source during an acceptable illumination condition. A method of facilitating operation of the flexible feeding tube with an imaging assembly including an imaging device and an illumination source and a controller is also disclosed. The imaging assembly is configured to generate imaging signals indicative of images of an alimentary canal of a subject. The illumination source is configured to illuminate an ambient environment of the imaging assembly. The controller is configured to receive the imaging signals and control the illumination source during an acceptable illumination condition. The method involves energizing the illumination source when an acceptable illumination condition is present and de-energizing the illumination source when the acceptable illumination condition is not present.

### BACKGROUND

Aspects of the present invention generally relate to an imaging catheter and, particularly, to an imaging feeding tube having a illumination control during placement to facilitate thermal management.

Several medical procedures involve positioning a catheter, such as a feeding tube or endoscope, within a patient through the patient's nose, mouth, or other opening. In many procedures, accurately positioning the catheter is crucial to the success of the procedure and/or to the safety of the patient. For example, a nasogastric feeding tube may be inserted through the nose, past the throat, and down into the stomach, or past the stomach into the small bowels of the patient to deliver food to the patient via the tube. If the feeding tube is mistakenly positioned in the patient's lung, the feeding solution would be delivered to the patient's lung causing critical and possibly fatal results.

### SUMMARY

One or more aspects of the invention can be directed to a feeding tube assembly. The feeding tube assembly can comprise a flexible feeding tube having opposite first and second longitudinal ends, a longitudinal axis extending between the first and second longitudinal ends, and a feeding passage defined therein extending along the longitudinal axis between the first and second longitudinal ends; an imaging assembly including an imaging device and an illumination source, the imaging assembly configured to generate and transmit imaging signals indicative of images of an alimentary canal of a subject, wherein the imaging assembly is secured to the feeding tube adjacent the first longitudinal end of the feeding tube, the illumination source being configured to illuminate an ambient environment of the imaging assembly; and a controller in communication with the illumination source, the controller configured to receive the imaging signals and to energize the illumination source during an acceptable illumination condition. In some embodiments pertinent to one or more aspects of the invention, the acceptable illumination condition terminates when the feeding tube is disconnected from the controller. In other embodiments pertinent to one or more aspects of the invention, the acceptable illumination condition terminates when a tube error occurs. In yet other embodiments pertinent to one or more aspects of the invention, the acceptable illumination condition terminates when an elapsed time during which the illumination source is energized is greater than or equal to a predetermined period. In particular embodiments, the predetermined period is 120 minutes. In still other embodiments pertinent to one or more aspects of the invention, the controller is further configured to determine if the illumination source is in condition for use. In further embodiments, the feeding tube assembly further comprises an inlet adaptor adjacent the second longitudinal end of the feeding tube in fluid communication with the feeding passage, the inlet adaptor configured for fluid connection with a source of enteral feeding liquid. The imaging assembly can comprise a flex circuit and the imaging device can be mounted on the flex circuit. In yet further embodiments, the imaging device has a field of view that is at least partially coincident with the illuminated ambient environment.

One or more aspects of the invention can be directed to a method of using a flexible feeding tube having opposite first and second longitudinal ends, a longitudinal axis extending between the first and second longitudinal ends, and a feeding passage defined therein extending along the longitudinal axis between the first and second longitudinal ends; an imaging assembly including an imaging device and an illumination source, the imaging assembly to generate imaging signals indicative of images of an alimentary canal of a subject, wherein the imaging assembly is secured to the feeding tube adjacent the first longitudinal end of the feeding tube, the illumination source being configured to illuminate an ambient environment of the imaging assembly; and a controller in communication with the illumination source, the controller configured to control the illumination source during an acceptable illumination condition. The method can comprise steps of, if an acceptable illumination condition is present; energizing the illumination source; and if the acceptable illumination condition is not present, de-energizing the illumination source. The method can further comprise determining an elapsed illumination time of energizing the illumination source, and wherein the acceptable illumination condition is not present when the elapsed illumination time is greater than or equal to a predetermined period. The predetermined period, in some embodiments, can be 120 minutes. In one or more embodiments, the acceptable illumination condition is not present when a tube error is determined. In one or more further embodiments the method the acceptable illumination condition is not present when the tube is disconnected from the controller.

Other objects and features will be in part apparent and in part pointed out hereinafter.

### BRIEF DESCRIPTION OF THE DRAWINGS

FIG. 1 is a schematic illustration showing a perspective view of an imaging feeding tube assembly in accordance with one or more aspects of the invention;

FIG. 2 is schematic illustration showing a perspective view of the imaging feeding tube assembly in FIG. 1, in accordance with one or more aspects of the invention;

FIG. 3 is a schematic illustration showing a side, elevational view of an imaging feeding tube system, including the imaging feeding tube assembly in FIG. 1, and an interface cable, and a console, in accordance with one or more aspects of the invention;

FIG. 4 is a schematic illustration showing an enlarged, fragmentary, perspective view of a distal end portion of the feeding tube assembly in FIG. 1, including an exploded imaging assembly, an imaging assembly connector, and a portion of the feeding tube, in accordance with one or more aspects of the invention;

FIG. 5 is a schematic illustration showing an enlarged cross section view of the feeding tube of the feeding tube assembly in FIG. 1, in accordance with one or more aspects of the invention;

FIG. 6 is a schematic illustration showing a top perspective view of a flex circuit assembly of the imaging assembly in FIG. 4, in a folded configuration, in accordance with one or more aspects of the invention;

FIG. 7 is a schematic illustration showing a bottom perspective view of the flex circuit assembly of the imaging assembly in FIG. 4, in the folded configuration, in accordance with one or more aspects of the invention;

FIG. 8 is a schematic illustration showing an enlarged fragmentary section view of the distal end portion of the imaging assembly in FIG. 4, in accordance with one or more aspects of the invention;

FIG. 9 is an electrical schematic of a thermal management system of the imaging assembly, in accordance with one or more aspects of the invention;

FIG. 10 is a block diagram schematically illustrating a controller configured to control an illumination source, in accordance with one or more aspects of the invention; and

FIG. 11 is a flow chart of a control routine of an imaging feeding tube assembly, in accordance with one or more aspects of the invention.

Corresponding reference characters indicate corresponding parts throughout the drawings.

### DETAILED DESCRIPTION

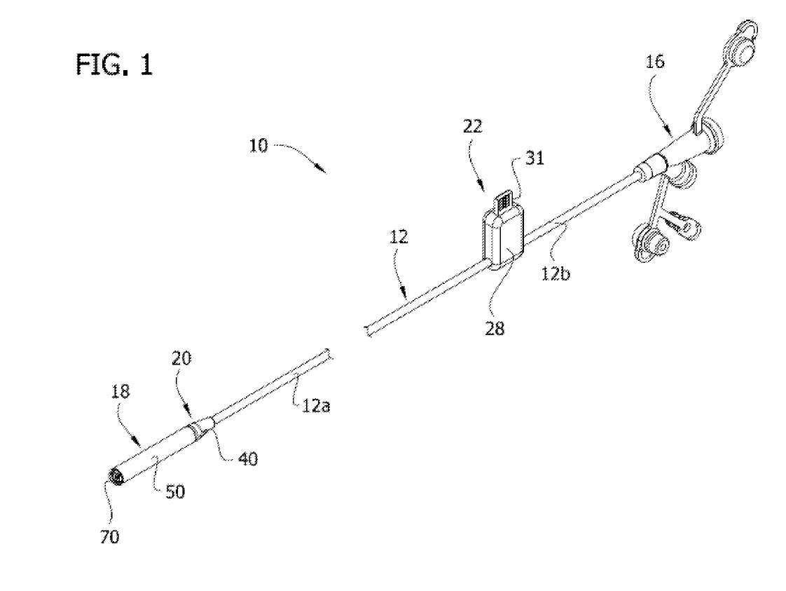

Referring now to the drawings, and in particular to FIGS. 1-3, an imaging catheter is generally indicated at 10. As disclosed herein, the imaging catheter can be a medical device that is configured for insertion into a subject (e.g., a human or a non-human subject) and configured to provide images (e.g., digital video) of anatomy of the subject as the medical device is inserted into the subject and/or after the medical device is positioned in the subject. In the illustrated embodiment, the imaging catheter is configured as a feeding tube assembly 10 and exemplarily illustrated as a nasogastric feeding tube assembly. In general, the illustrated nasogastric feeding tube assembly 10 can be configured to provide images of an alimentary canal, or a portion(s) thereof, of the subject as the feeding tube assembly is inserted into the subject and after the feeding tube assembly is positioned in the subject to facilitate confirmation of proper placement of the feeding tube assembly in the subject. The nasogastric feeding tube assembly 10 can be also configured to deliver liquid nutrients into the alimentary canal of the subject by enteral feeding, such as after a user (e.g., medical practitioner) confirms proper placement of the feeding tube assembly in the subject, by viewing the acquired digital images from the imaging feeding tube assembly. It is understood that the imaging catheter 10 may be configured as a different type of feeding tube, such as a gastric feeding tube, or a jejunostomy feeding tube, or may be configured as a different type of medical device, such as an endoscope, or a heart catheter (e.g., balloon catheter or other type of heart catheter).

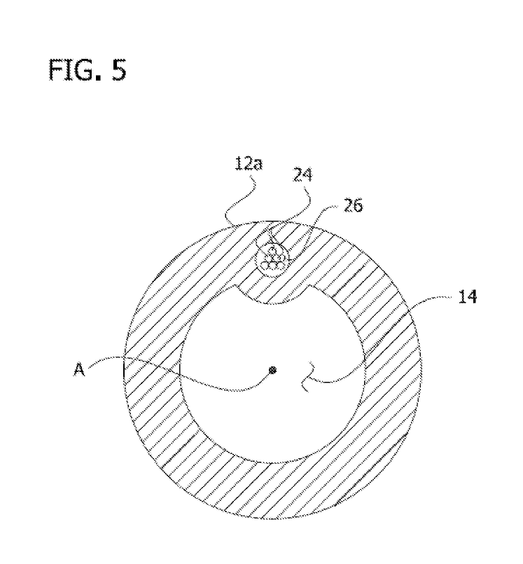

The illustrated feeding tube assembly 10 generally includes an elongate, generally flexible body in the form of a feeding tube, generally indicated at 12, having a longitudinal axis A (FIG. 5), an open first longitudinal end (i.e., a distal end) and an open second longitudinal end (i.e., a proximal end). A feeding passage 14, defined by an interior surface of the feeding tube 12, extends longitudinally between the longitudinal ends of the tube for delivering nutrients (e.g., in the form of an enteral feeding solution) to the subject. In other embodiments—such as catheters that are not feeding tubes—the elongate body may have other configurations, and may not have a longitudinal passage for delivering fluids to the patient. An inlet adapter, generally indicated at 16, for delivering liquid nutrients into the feeding passage 14 is attached to the second end of the tube, and an imaging assembly, generally indicated at 18, for generating and transmitting real time images (e.g., video) of the alimentary canal of the patient during and/or following intubation is attached to the first end of the tube 12 by an imaging assembly connector, generally indicated at 20.

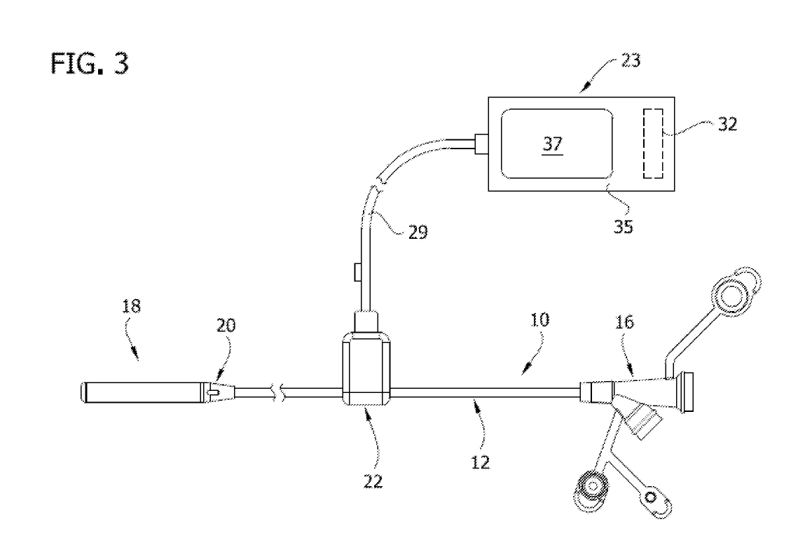

As used herein with the point of reference being the feeding source, the inlet adaptor 16 defines the proximal end of the feeding tube assembly 10, and the imaging assembly 18 defines the distal end. The feeding tube assembly 10 can also include a console connector, generally indicated at 22, in communication with the imaging assembly 18, to provide communication between the imaging assembly and a console 23 (FIG. 3), on which the images obtained by the imaging assembly 18 may be displayed. In the illustrated embodiment, the feeding tube assembly 10, the console 23, and an interface cable 29, which communicatively connects the feeding tube assembly to the console, together constitute an imaging catheter system, and more specifically, an imaging feeding tube system.

Referring to FIGS. 1-3, the exemplarily illustrated feeding tube 12 comprises two tube segments: a first tube segment 12a extending between the imaging assembly connector 20 and the console connector 22, and a second tube segment 12b extending between the console connector and the inlet adaptor 16. The first and second tube segments 12a, 12b can be secured to the console connector 22 in such a way that the first and second tube segments are in fluid communication with each other to at least partially define the feeding passage 14. In other embodiments of the disclosure, the tube 12 may be formed as an integral, one-piece component. The feeding tube 12 may be formed from a thermoplastic polyurethane polymer, such as but not limited to, an aromatic, polyether-based thermoplastic polyurethane, and a radiopaque substance, such as barium. The tube 12 may be formed from other materials without departing from the scope of the present disclosure.

As shown in FIG. 5, the first tube segment 12a of the feeding tube 12 may include one or more electrical conductors 24 typically disposed in the tube wall of the first tube segment. The electrical conductors 24 run longitudinally along the first tube segment, such as along or parallel a longitudinal axis of the feeding passage 14. At least some of the electrical conductors 24 can be configured to transmit imaging signals between the imaging assembly 18 and the console 23. Other electrical conductors 24 may be configured to transmit power from the console 23 to the imaging assembly 18, and provide a ground. Still other electrical conductors 24 may be configured to provide other communication including, but not limited to, two-way communication, between the console 23 and the imaging assembly 18. In one or more embodiments of the disclosure, at least one of the electrical conductors 24 is configured to supply power from a power supply, which can be the console 23, to the imaging assembly 18, although other ways of powering the imaging assembly, including the imaging assembly having its own source of power, do not depart from the scope of the present disclosure. As exemplarily illustrated, the electrical conductors 24 can be disposed within a conductor passage 26 of the feeding tube 12 so that the conductors are physically separated or at least fluidly isolated from the feeding passage 14 to inhibit or reduce the likelihood of feeding solution in the feeding passage from contacting the conductors.

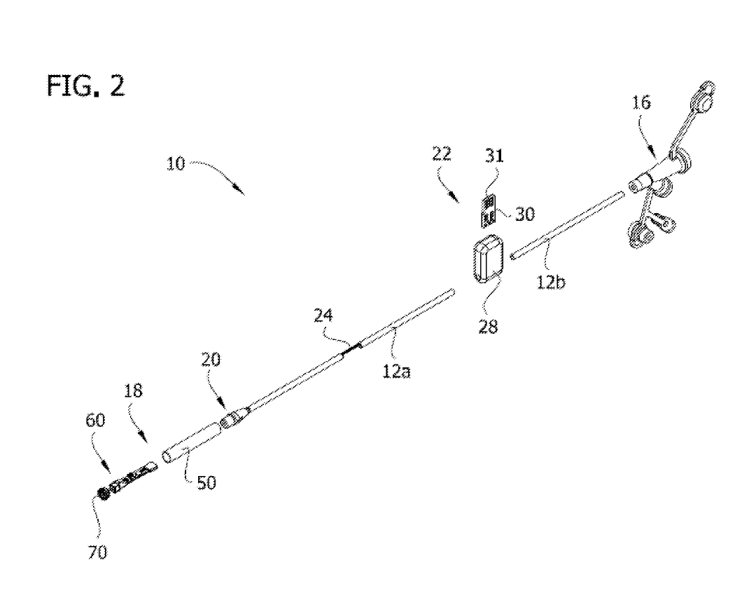

The electrical conductors 24 extend from the first tube segment 12a into a connector housing 28 of the console connector 22 and are electrically connected to a PCB 30 (FIG. 2). The interface cable 29 (or other signal-transmitting component) can be removably connectable to edge connector 31 to effect communication and data exchange between the console 23 and the imaging assembly 18. An electronic memory component, such as electrically erasable programmable read-only memory (EEPROM), may be mounted on the PCB 30 to allow information (i.e., data) to be stored and/or written thereon and to be accessible by the console 23 (i.e., a controller 32 of the console 23) or another external device. It is understood that the PCB 30 may have additional or different electrical components mounted thereon, or the PCB may be omitted such that the electrical conductors are operatively connected to the

PCB 30. The console 23 can also include a console housing 35, a console display 37, such as an LCD or other electronic display, secured to the housing, and controller 32 (broadly, “a control circuit” or “controller”) disposed in the housing. In the illustrated embodiment, the controller 32 communicates with the imaging assembly 18 through the interface cable 29 and the electrical conductors 24.

Referring to FIGS. 1, 2, and 4 the imaging assembly connector 20 may define a feeding outlet 40 that is in fluid communication with the feeding passage 14 of the tube 12. The feeding outlet 40 can comprise one or more openings extending laterally through a side of the imaging assembly connector 20 (only one such lateral opening is illustrated). In the illustrated embodiment, the first or distal end of the tube 12 is received and secured within the imaging assembly connector 20 at a proximal end of the imaging assembly connector to provide fluid communication between the feeding passage 14 and the feeding outlet 40. When the feeding tube assembly 10 is determined to be appropriately positioned in a patient, feeding solution or other desirable liquid fed into the inlet adaptor 16 can be introduced through the feeding passage 14 of the tube 12, and out through the outlet 40 and into the subject's alimentary canal.

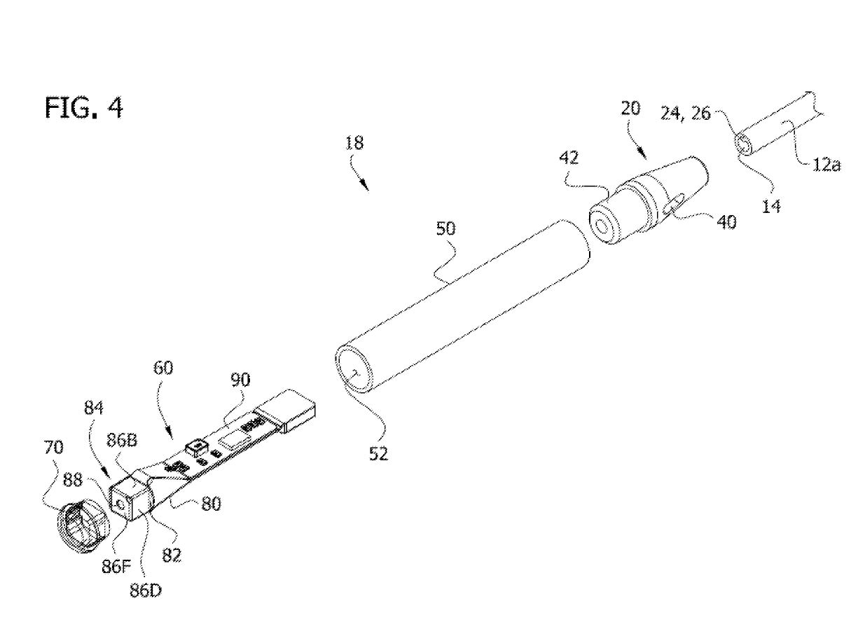

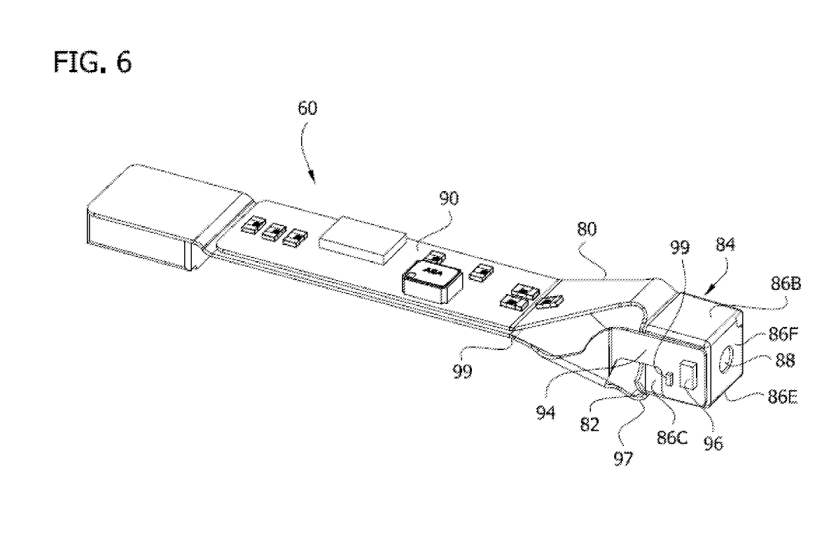

Referring to FIG. 4, the imaging assembly 18 can include a tubular housing 50, a flexible circuit (“flex circuit”) assembly 60 disposed within the tubular housing, and a transparent or translucent cap 70 secured to the tubular housing 50. Generally speaking a flex circuit includes a deformable circuit element and components mounted on the deformable circuit element. The deformable circuit element may be a flat (at least prior to being deformed) substrate that can be bent or otherwise deformed, and which also includes electrical conductors for making electrical connection among various components that may be mounted on the substrate. The deformable circuit element may only be partially deformable (e.g., only at discrete bend lines) within the scope of the present disclosure. Among other functions, the tubular housing 50 can provide protection for the flex circuit assembly 60, and the housing may be substantially waterproof to inhibit the ingress of liquid into the imaging assembly 18. The tubular housing 50 has an interior surface defining an axial passage 52 shaped and sized for housing the flex circuit assembly 60 in a folded configuration. In one embodiment, the tubular housing 50 is formed from a generally flexible material that provides protection for the flex circuit assembly 60 and allows the imaging assembly 18 to bend to facilitate maneuverability of the feeding tube assembly 10. A second end, such as a proximal end, of the tubular housing 50 can be configured to receive a connection portion 42 of the imaging assembly connector 20, and can be adhered thereto to secure the imaging assembly to feeding tube 12. The tubular housing 50 may be generally opaque, by being formed from an opaque white material or having an opaque material applied thereon, to reflect illumination from a light source or illumination source, such as an internal LED 96 (FIG. 6), and direct the illumination outward from the distal end of the imaging assembly 18 to, for example, a field of view.

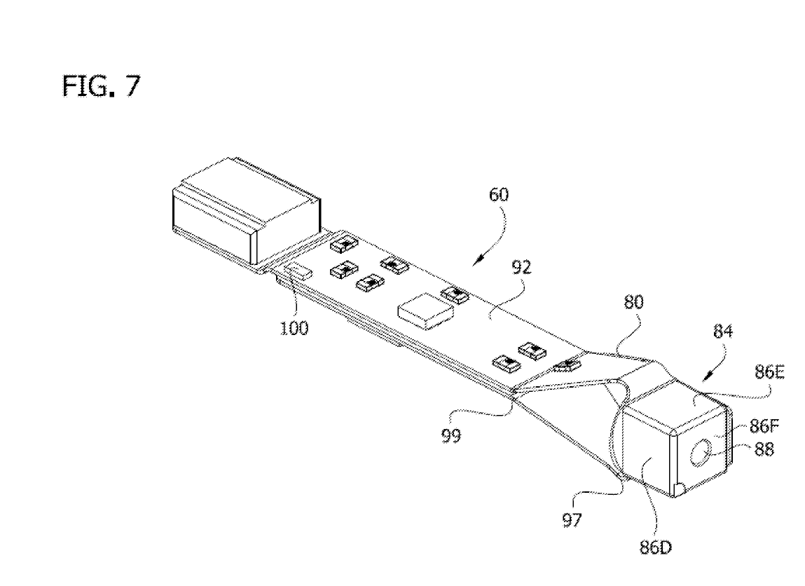

The flex circuit assembly 60 typically includes a flex circuit 80 and electronic components (not labeled), described below, attached thereto. In the partially assembled or folded configuration exemplarily shown in FIGS. 4, 6, and 7, the flex circuit assembly 60 can have a length with a first longitudinal end, e.g., a distal end, and an opposite second longitudinal end, e.g., a proximal end. The electrical conductors 24 can be connected to the second longitudinal end, e.g., the proximal end, of the flex circuit assembly 60. A camera mounting portion 82 is typically disposed at the first longitudinal end, e.g., the distal end of the flex circuit assembly 60. An imaging device such as a digital camera, generally indicated at 84, can be mounted on the camera mounting portion 82. The camera 84 can have a cuboidal housing 86 with a base 86A, as shown in FIG. 7, sides 86B, 86C, 86D, 86E, and an upper or first surface 86F. The distal end surface 86F of the camera 84 can include a lens 88. The lens 88 defines a field of view that projects generally outward from the distal end of the imaging assembly 18. In accordance with one or more embodiments of the disclosure, the camera 84 comprises an imaging device, such as a CMOS imaging device. In further embodiments of the disclosure, the camera 84 may comprise a different type of solid state imaging device, such as a charge-coupled device (CCD), or another type of imaging device. Other ways of configuring the electronics and other components of the imaging assembly 18 do not depart from the scope of the present disclosure and may be implemented as variant embodiments thereof.

For example, in another embodiment, the flex circuit assembly 60 may be replaced with a rigid printed circuit board (PCB). Moreover, it will be understood that an optical imaging assembly (not shown) may be used.

The flex circuit assembly 60 can include a power mounting portion 90 (FIGS. 4 and 6) and a control or data mounting portion 92 (FIG. 7) each typically extending from the camera mounting portion 82 at a fold line toward the first longitudinal end of the flex circuit assembly 60. Power supply components are typically disposed on the power mounting portion 90, and camera control components are typically disposed on the data mounting portion 92.

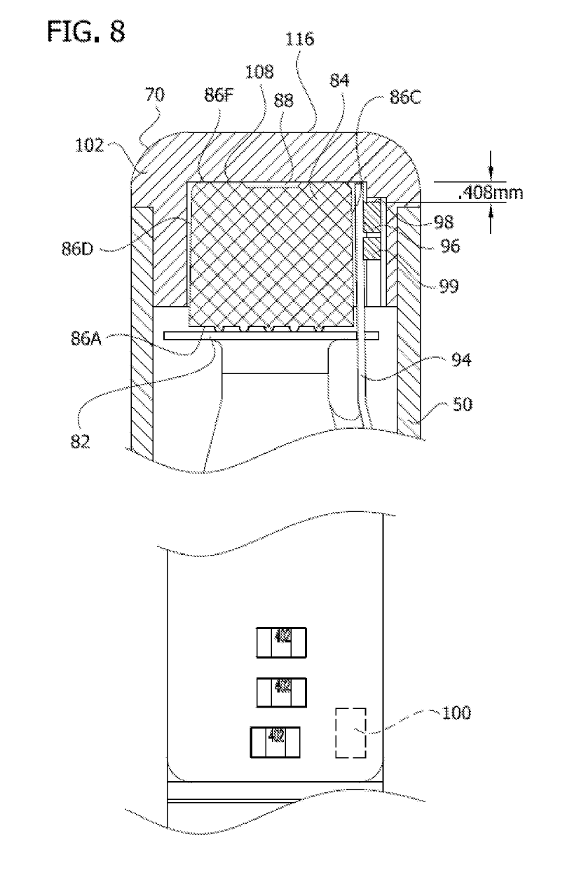

Referring to FIGS. 6 and 8, a light mounting portion 94 of the flex circuit 60 can be disposed at the side 86C of the camera 84. The light mounting portion 94 is illustratively depicted as extending longitudinally toward the camera 84 from a lateral side edge of the flex circuit at a fold line of the power mounting portion 90. One or more light sources 96 can be disposed on, for example, the light mounting portion 94 for illuminating an area or region adjacent to the distal end surface 86F of the camera housing 86. In the illustrated embodiment, the light source is a light emitting diode (LED) 96 disposed on the light mounting portion 94 so that the LED is disposed on the side 86C of the camera housing and below or proximate the upper surface 86F of the camera housing. In the illustrated embodiment, the LED 96 has a light emitting surface 98 substantially perpendicular to the light mounting portion 94 for projecting light outward from the distal end of the imaging assembly 18. According to the illustrated embodiment (FIG. 8), the LED and the light mounting portion 94 are positioned relative to the camera 84 and the camera mounting portion 82 such that the light emitting surface 98 of the LED 96 is a relatively short distance (e.g., 0.408 millimeters) below the upper surface 86F of the camera housing 86. Typically, LED 96 has an illumination zone that is at least partially coincident over an imaging zone or field of view of camera 84, through optional lens 88.

A light source temperature sensor 99 may be disposed on the light mounting portion 94 adjacent the LED 96. The light source temperature sensor 99 is configured to measure a temperature of the LED 96. An ambient temperature sensor 100 may be disposed on the control mounting portion 92. However, it is envisioned that the ambient temperature sensor 100 could be disposed at other locations on or adjacent the flex circuit assembly 60. The ambient temperature sensor 100 is configured to measure a temperature of the ambient environment around the imaging assembly 18. As will be explained in greater detail below, measuring both the light source temperature and the ambient temperature allows for a determination of the difference between the two temperatures for regulating the difference between the two temperatures during use of the imaging catheter 10.

Operation of the LED 96 to illuminate the field of view of lens 88 may cause the temperature of the LED to exceed that of the ambient environment around the imaging assembly 18 by more than a desirable amount. To ensure the difference between the ambient temperature and the light source temperature does not fluctuate away from an acceptable amount while maintaining the maximum output of light for viewing, controller 32 may selectively control an output of the LED 96. In particular, the controller 32 may control the output of the LED 96 by controlling the power supplied by a power source (e.g., console 23) to the LED. A PWM driver may also be used to drive the LED 96 and the controller 32 may control the PWM driver to control the output of the LED.

As mentioned above, controlling the output of the LED 96 may be used to control the difference between the ambient temperature and light source temperature detected by the ambient temperature sensor 100 and light source temperature sensor 99, respectively. For example, if the temperature sensors 99, 100 detect respective temperatures having a difference other than a predetermined amount, the controller 32 can adjust (i.e., increase or decrease) the output of the LED 96 to regulate the temperature difference between the ambient environment around the imaging assembly 18 and the LED 96. Alternatively, the controller 32 may continually control the power supplied to the LED 96 to continually control the output of the LED so that the difference between the ambient temperature and the light source temperature remains at a predetermined amount. In this instance, power can be increased and decreased as needed to keep the difference between the ambient temperature and the light source temperature at the predetermined amount. The controller 32 can include a control loop mechanism such as a PID controller to maintain the difference between the ambient environment and the LED 96 at the predetermined amount. In it envisioned that both analog and digital control loops can be used within the scope of the present disclosure.

In a preferred embodiment the controller 32 maintains the temperature difference between the ambient environment and the LED 96 to about 2 degrees Celsius. The controller may alternatively maintain the temperature difference within a predetermined range. The range may be centered on a temperature difference of about 2 degrees Celsius. The controller 23 may maintain the temperature difference at other values within the scope of the present disclosure. In some cases, the temperature difference is about 1 degree Celsius.

In a situation where the imaging catheter is operating in a relatively cold environment or an environment where there is relatively little heat transfer from the LED 96, the difference between the temperature of the LED and the ambient temperature around the imaging assembly 18 may increase above the predetermined amount. In this instance the temperature difference is a positive difference where the temperature of the LED 96 is greater than the ambient temperature. Should the temperature difference exceed the predetermined amount, the controller 32 may decrease the output of the LED 96 to decrease the temperature of the LED to restore the desired temperature difference between the LED and the ambient environment.

If the imaging assembly 18 enters an area of the body that provides a substantial heat sink for the LED 96, the LED temperature detected by the LED temperature sensor 99 may fall below the predetermined temperature differential and possibly even below the ambient temperature detected by the ambient temperature sensor 100. The controller 32 may increase the output of the LED 96 up to a maximum output to permit the most light possible for viewing, while monitoring any resultant temperature change in the LED temperature sensor 99. It will be understood that the output of the LED 96 can be increased while still maintaining the temperature difference at the predetermined amount. This allows the imaging catheter 10 to operate with a maximum light permissible output from the LED 96 at all times.

The amount in which the temperature difference exceeds the desired amount may also control the rate and/or extent to which the output of the LED 96 is increased or decreased. Thus, a large temperature difference above or below the desired amount may result in a significant increase or decrease in the output of the LED 96. In the instance where a significant decrease in the output of the LED 96 is required, the controller 23 may completely shut off the output of the LED (i.e., turn off all power to LED). Conversely, when a significant increase in the output of the LED 96 is required, the controller 23 may supply maximum power to the LED.

In some embodiments, the controller 32 may reduce the power supplied to the LED to reduce the output of the LED in order to reduce the ambient temperature around the imaging assembly 18 if the ambient temperature sensor 100 detects an ambient temperature above a predetermined threshold. Alternatively, the controller 32 may continually control the power supplied to the LED 96 to continually control output of the LED so that the ambient temperature remains below the predetermined threshold. In this instance, power can be increased and decreased as needed to keep the ambient temperature below the predetermined threshold. In a preferred embodiment, the controller 32 controls the output of the LED 96 to a maximum output of the LED (i.e., maximum power supplied to the LED) as long as the ambient temperature measured by the ambient temperature sensor 100 remains below the predetermined threshold.

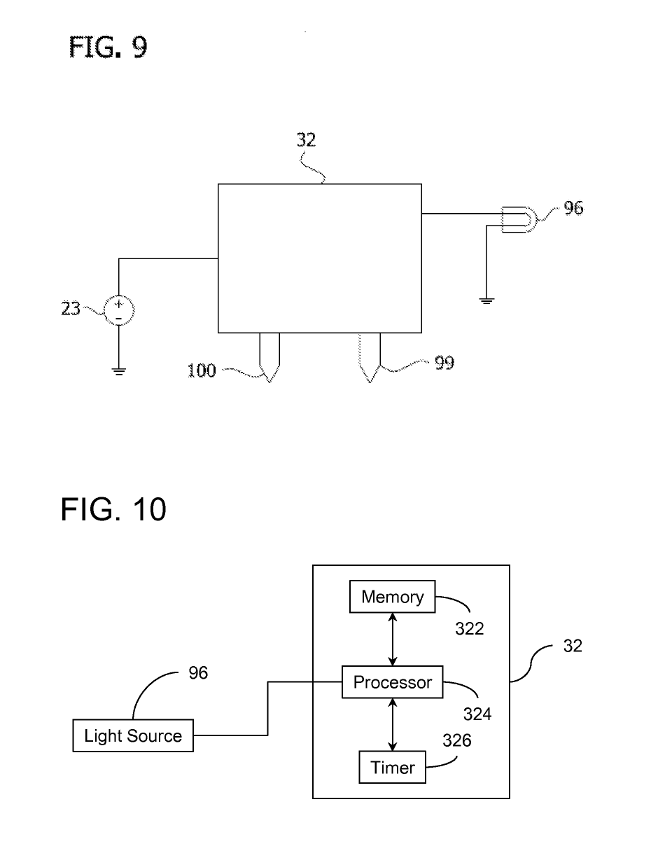

In the illustrated embodiment, the temperature sensors 99, 100 are thermistors. However, other types of temperature sensors are envisioned. Further, it is envisioned that a single temperature sensor can be used to measure both the ambient temperature and the temperature of the LED 96. The console (i.e., power supply) 23, controller 32, LED 96, light source temperature sensor 99, and ambient temperature 100 may be broadly considered a thermal management system (FIG. 9).

The feeding tube assembly can be configured to regulate the operation of a heat generating component, such as the illumination source to be operable during an acceptable operating condition, such as an operating illumination condition. Such aspects of the invention can facilitate the use of the assembly to reduce the risk of harm on the patient during, for example, insertion procedures or post-insertion procedures of the feeding tube assembly. In some cases, the risk of harm is reduced during or after prolonged insertion of the assembly in the patient by de-energizing heat generating components of the assembly when disposed inside the patient. For example, in some embodiments pertinent to one or more aspects of the invention, the controller 32 can further control the operation of the illumination source.

The feeding tube assembly can comprise a flexible feeding tube; an imaging assembly including an imaging device and an illumination source. The imaging assembly has a field of view and can be configured to generate and transmit imaging signals indicative of images of the field of view of an alimentary canal of a subject. The imaging assembly can be secured to the feeding tube adjacent a first longitudinal end of the feeding tube. The illumination source can be configured to illuminate an ambient environment of the imaging assembly. The field of view is at least partially coincident with the illuminated ambient environment. The assembly further has a controller in communication with the illumination source and configured to receive the imaging signals representative of the field of view. The controller is configured to energize the illumination source during an acceptable illumination condition. The acceptable illumination condition terminates when the feeding tube is disconnected from the controller. In other embodiments pertinent to one or more aspects of the invention, the acceptable illumination condition terminates when a tube error occurs. In yet other embodiments pertinent to one or more aspects of the invention, the acceptable illumination condition terminates when an elapsed time during which the illumination source is energized is greater than or equal to a predetermined period. In still other embodiments pertinent to one or more aspects of the invention, the controller is further configured to determine if the illumination source is in condition for use. In further embodiments, the feeding tube assembly further comprises an inlet adaptor adjacent the second longitudinal end of the feeding tube in fluid communication with the feeding passage, the inlet adaptor configured for fluid connection with a source of enteral feeding liquid. The imaging assembly can comprise a flex circuit and the imaging device can be mounted on the flex circuit.

With reference to FIG. 10, the controller 32 can comprise one or more processors 324, such as a microprocessor, operably configured to control the illumination source 96, such as by energizing or controlling or regulating the amount of power delivered to energize the illumination source 96. The processor 324 is also operably connected to a non-transitory memory device 322 having instructions stored thereon that are retrievable and executable by the one or more processors 324. The processor can also be operably connected a timer device or clock or timer 326 configured to receive a signal from the one or more processors 324. In some cases, the timer 326 is further configured to send one or more signals to the processor 324. Alternatively, the processor can have an internal clock that determines an elapsed time.

The one or more of the memory devices 322 can store data, for example, in a data storage segment thereof (not shown). The data that can be stored can be, for example, image signals in data files that are representative of images captured by the imaging device. Data in the data storage segment can also include one or more operating conditions or parameters of the feeding tube assembly.

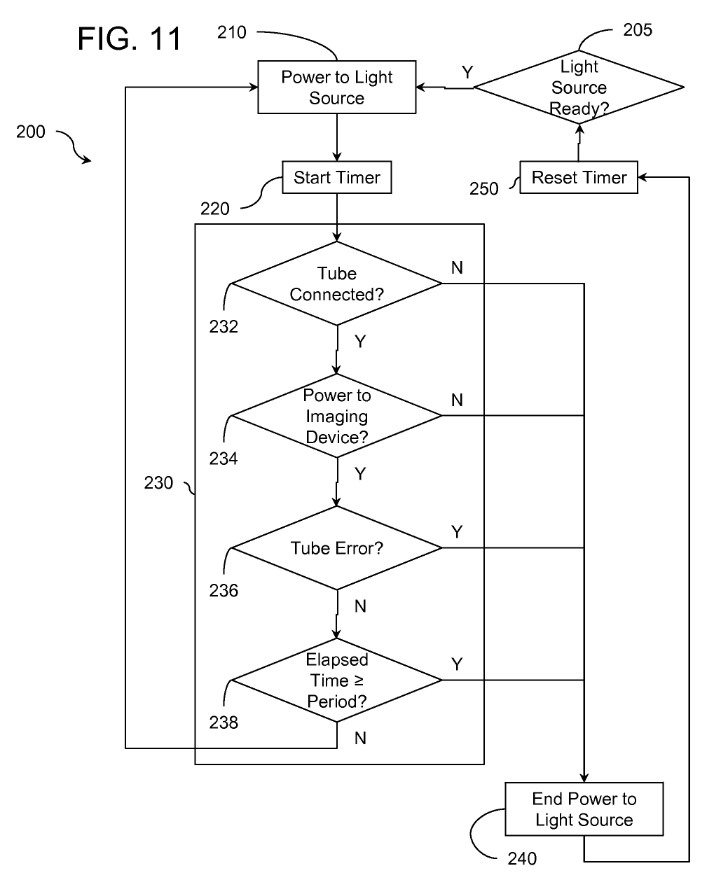

The use of the feeding tube assembly can involve facilitating use of the flexible feeding tube with the imaging assembly. As exemplarily illustrated in FIG. 11, facilitating use can involve operation of the feeding tube assembly according to a control algorithm 200 that manages operation of any heat generating source of the imaging assembly, such as the illumination source 96. The algorithm can be stored as instructions on one or more of the memory devices 322 and can comprise steps of, if an acceptable illumination condition is present; energizing the illumination source; and if the acceptable illumination condition is not present, de-energizing the illumination source.

In the illustrated example, the algorithm 200 can involve determining 205 if the light source or the feeding tube, or both, is ready for use. For example, the tube is ready for use if the timer 326 provides an indication that a counter or in-use period of the illumination source is at an initial condition. Other precondition requirements can involve determining whether the feeding tube is properly connected, or provides no error signals, flags, or indications. Use of the assembly can then be initiated by a user by an initiation signal (not shown). The controller 32 can then send instructions to energize or provide 210 power to the illumination source 96 and to send 220 a time initiation signal to commence measuring an elapsed time of operation of the illumination source 96. The controller monitors or controls operation of the illumination source 96 during an acceptable condition and determines 230 that the acceptable illumination condition remains present. In a pertinent embodiment, the step of determining 205 if the illumination source is ready (or determines if the precondition requirements are acceptable) involves substantially similar determinations of conditions as those in step 230. In some cases, determining 230 if the acceptable illumination conditions remain present can involve any one or more steps of determining 232 if the feeding tube is properly connected, determining 234 whether power is still delivered to the tube and the imaging device, determining 236 whether any errors have been received from the feeding tube, and determining 238 whether the elapsed time, e.g., the elapsed illumination time, is greater than or equal to a predetermined operating period.

If the acceptable illumination period is no longer present, or the precondition is not acceptable, the controller terminates 240 power to de-energize the illumination source. After terminating the power to the illumination source, the timer can be reset 250 after a reset period has elapsed. The reset period can be a predefined reset period, such as at least about one hour of non-use. Any of the predetermined period and the predefined reset period can be two hours, but in other cases, any of the periods can be sixty minutes or ninety minutes. Typically, the periods are different; thus, for example, the predetermined period for termination of the acceptable condition can be 120 minutes but the predefined reset period after termination and prior to initiation can be 6 hours, 12 hours, or 24 hours. As with the predetermined period, the predefined reset period can be stored in one or more of the memory devices 322 and be retrievable by the one or more processors 324. The method can further include initiating (not shown) one or more visual and audible alarms when the acceptable illumination condition is not present.

Further embodiments can also include providing one or more indications, such as one or more visible and/or audible messages when the elapsed time during which the illumination source is energized approaches the predetermined period. For example, a message can be presented on the console display 37 that notifies a user that the timer is at 90 minutes or at a percentage, e.g., at or less than 10% of the predetermined period.

In other cases, the timer 326 continues determining the elapsed illumination period but suspends (not shown) determination of the elapsed illumination period when power to the illumination source is interrupted. The timer resumes determination of the elapsed illumination period when the illumination source is re-energized.

In further cases, the timer 326 is reset 250 or the acceptable illumination condition becomes present when the existing connected feeding tube is disconnected and a second, different feeding tube is connected to the console 23.

In other cases, the timer 326 is reset 250 or the acceptable illumination condition becomes present when the console 23 is powered down. In some cases, however, the acceptable illumination condition becomes present only after the predefined reset period is elapsed, even when the console 23 is off.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions and methods without departing from the scope of the invention, it is intended that all matter contained in the above description and shown in the accompanying drawings shall be interpreted as illustrative and not in a limiting sense.

1. A feeding tube assembly comprising:
a flexible feeding tube having opposite first and second longitudinal ends, a longitudinal axis extending between the first and second longitudinal ends, and a feeding passage defined therein extending along the longitudinal axis between the first and second longitudinal ends;
an imaging assembly including an imaging device and an illumination source, the imaging assembly configured to generate and transmit imaging signals indicative of images of an alimentary canal of a subject, wherein the imaging assembly is secured to the feeding tube adjacent the first longitudinal end of the feeding tube, the illumination source being configured to illuminate an ambient environment of the imaging assembly; and
a controller in communication with the illumination source, the controller configured to receive the imaging signals and to energize the illumination source during an acceptable illumination condition.

2. The feeding tube assembly set forth in claim 1, wherein the acceptable illumination condition terminates when the feeding tube is disconnected from the controller.

3. The feeding tube assembly set forth in claim 1, wherein the acceptable illumination condition terminates when a tube error occurs.

4. The feeding tube assembly set forth in claim 1, wherein the acceptable illumination condition terminates when an elapsed time during which the illumination source is energized is greater than or equal to a predetermined period.

5. The feeding tube assembly set forth in claim 1, wherein the controller is further configured to determine if the illumination source is in condition for use.

6. The feeding tube assembly set forth in claim 1, further comprising an inlet adaptor adjacent the second longitudinal end of the feeding tube in fluid communication with the feeding passage, the inlet adaptor configured for fluid connection with a source of enteral feeding liquid.

7. The feeding tube assembly set forth in claim 1, wherein the imaging assembly comprises a flex circuit, and wherein the imaging device is mounted on the flex circuit.

8. The feeding tube assembly set forth in claim 1, wherein the imaging device has a field of view that is at least partially coincident with the illuminated ambient environment.

9. A method of regulating thermal performance of a flexible feeding tube having opposite first and second longitudinal ends, a longitudinal axis extending between the first and second longitudinal ends, and a feeding passage defined therein extending along the longitudinal axis between the first and second longitudinal ends; an imaging assembly including an imaging device and an illumination source, the imaging assembly configured to generate imaging signals indicative of images of an alimentary canal of a subject, wherein the imaging assembly is secured to the feeding tube adjacent the first longitudinal end of the feeding tube, the illumination source being configured to illuminate an ambient environment of the imaging assembly; and a controller in communication with the illumination source, the controller configured to receive the imaging signals and to control the illumination source during an acceptable illumination condition, the method comprising:
if an acceptable illumination condition is present; energizing the illumination source; and
if the acceptable illumination condition is not present, de-energizing the illumination source.

10. The method of claim 9, further comprising determining an elapsed illumination time of energizing the illumination source, and wherein the acceptable illumination condition is not present when the elapsed illumination time is greater than or equal to a predetermined period.

11. The method of claim 10, wherein the predetermined period is 120 minutes.

12. The method of claim 9, wherein the acceptable illumination condition is not present when a tube error is determined.

13. The method of claim 9, wherein the acceptable illumination condition is not present when the tube is disconnected from the controller.
